SEQUENCE LISTING (82 sequences)

SEQ ID NO: 1 - DNA, 35 bp, chicken
aagacagcaccaggacacaaataaataaggtgagc

SEQ ID NO: 2 - DNA, 35 bp, chicken
aaggtgagcctacagttaaagattaaaacctttgc

SEQ ID NO: 3 - DNA, 35 bp, chicken
aagattaaaacctttgccctgctcaatggagccac

SEQ ID NO: 4 - DNA, 35 bp, chicken
aagtgtggccacctccaactcccagagtgttaccc

SEQ ID NO: 5 - DNA, 35 bp, chicken
aagctcaggtacagaaataatttcacctccttctc

SEQ ID NO: 6 - DNA, 35 bp, chicken
aagcaaaatacagcagatgaagcaatctcttagct

SEQ ID NO: 7 - DNA, 35 bp, chicken
aagcaatctcttagctgttccaagccctctctgat

SEQ ID NO: 8 - DNA, 35 bp, chicken
aagaaaaacagcacaaaattgtaaatattggaaaa

SEQ ID NO: 9 - RNA, 35 nt, Artificial Sequence
  crRNA
aagacagcaccaggacacaaauaaauaaggugagc

SEQ ID NO: 10 - RNA, 35 nt, Artificial Sequence
  crRNA
aaggugagccuacaguuaaagauuaaaaccuuugc

SEQ ID NO: 11 - RNA, 35 nt, Artificial Sequence
  crRNA
aagauuaaaaccuuugcccugcucaauggagccac

SEQ ID NO: 12 - RNA, 35 nt, Artificial Sequence
  crRNA
aaguguggccaccuccaacucccagaguguuaccc

SEQ ID NO: 13 - RNA, 35 nt, Artificial Sequence
  crRNA
aagcucagguacagaaauaauuucaccuccuucuc

SEQ ID NO: 14 - RNA, 35 nt, Artificial Sequence
  crRNA
aagcaaaauacagcagaugaagcaaucucuuagcu

SEQ ID NO: 15 - RNA, 35 nt, Artificial Sequence
  crRNA
aagcaaucucuuagcuguuccaagcccucucugau

SEQ ID NO: 16 - RNA, 35 nt, Artificial Sequence
  crRNA
aagaaaaacagcacaaaauuguaaauauuggaaaa

SEQ ID NO: 17 - DNA, 2664 bp, Artificial Sequence
  Cas 3 gene
atggaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgtgggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaa

SEQ ID NO: 18 - DNA, 672 bp, Artificial Sequence
  Cas 5 gene
atgcgctcctacctgatcctgagactggcaggaccaatgcaggcatggggacagcctacattcgagggaaccaggccaacaggccgctttcctacccggtctggactgctgggactgctgggagcctgcctgggcatccagagggacgatacctctagcctgcaggccctgagcgagtccgtgcagttcgccgtgcgctgtgatgagctgatcctggacgataggcgcgtgtccgtgacaggcctgcgggattaccacaccgtgctgggcgccagagaggactataggggcctgaagtcccacgagaccatccagacatggcgcgagtacctgtgcgacgcctcttttacagtggccctgtggctgaccccacacgcaacaatggtcatcagcgagctggagaaggccgtgctgaagccacggtacaccccctatctgggccggagaagctgccctctgacacacccactgttcctgggcacctgtcaggcctccgatccccagaaggccctgctgaactacgagcctgtgggcggcgacatctattctgaggagagcgtgacaggccaccacctgaagttcaccgccagggatgagccaatgatcacactgccaaggcagtttgcatccagggagtggtatgtgatcaagggaggaatggacgtgagccag

SEQ ID NO: 19 - DNA, 597 bp, Artificial Sequence
  Cas 6 gene
atgtacctgagcaaagtgatcatcgcaagggcatggtccagggacctgtatcagctgcaccagggcctgtggcacctgttccctaatagaccagatgccgccagggacttcctgtttcacgtggagaagaggaacacacccgagggctgtcacgtgctgctgcagtccgcccagatgcccgtgagcaccgcagtggccacagtgatcaagaccaagcaggtggagttccagctgcaagtgggcgtgccactgtactttaggctgcgcgccaatcccatcaagaccatcctggataaccagaagcgcctggactctaagggcaatatcaagcggtgcagagtgcctctgatcaaggaggccgagcagatcgcctggctgcagagaaagctgggcaacgccgccagggtggaggatgtgcaccctatcagcgagcggccacagtatttcagcggcgacggcaagtccggcaagatccagaccgtgtgctttgagggcgtgctgaccatcaacgatgccccagccctgatcgacctggtgcagcagggaatcggacctgctaagtcaatgggatgtgggctgctgtcactggcacctctg

SEQ ID NO: 20 - DNA, 1089 bp, Artificial Sequence
  Cas 7 gene
atgtccaatttcatcaacatccacgtgctgatctcccactctccaagctgcctgaatagagacgatatgaacatgcagaaggacgccatctttggcggcaagcggagagtgaggatctctagccagtccctgaagcgggccatgagaaagtctggctactatgcccagaatatcggcgagtcctctctgaggaccatccacctggcacagctgagggacgtgctgagacagaagctgggcgagcggtttgatcagaagatcatcgacaagacactggccctgctgagcggcaagtccgtggatgaggccgagaagatcagcgccgacgcagtgaccccatgggtggtgggagagatcgcatggttctgtgagcaggtggccaaggccgaggccgataatctggacgataagaagctgctgaaggtgctgaaggaggatatcgccgccatcagagtgaacctgcagcagggagtggacatcgccctgagcggcaggatggccacatccggcatgatgaccgagctgggcaaggtggacggagcaatgtccatcgcacacgccatcaccacacaccaggtggactctgatatcgactggttcacagccgtggacgatctgcaggagcagggaagcgcccacctgggaacccaggagttcagctccggcgtgttttacagatatgccaatatcaacctggcacagctgcaggagaacctgggaggagcatccagggagcaggccctggagatcgccacacacgtggtgcacatgctggcaaccgaggtgccaggagcaaagcagcgcacctacgccgccttcaatcctgccgatatggtcatggtgaacttttccgacatgccactgtctatggccaatgccttcgagaaggccgtgaaggccaaggatggcttcctgcagccttccatccaggcctttaaccagtactgggaccgcgtggccaatggatatggcctgaacggagctgccgcccagttttccctgtctgatgtggaccctatcacagcccaggtgaagcagatgccaaccctggagcagctgaagagctgggtgcggaacaatggagaggca

SEQ ID NO: 21 - DNA, 1506 bp, Artificial Sequence
  Cas 8 gene
atgaacctgctgattgacaactggatccctgtgcgcccacggaacggaggaaaagtccagattattaatctgcagagcctgtactgctcccgggatcagtggagactgagcctgcccagagacgatatggagctggccgccctggccctgctggtgtgcatcggccagatcatcgcccctgccaaggacgatgtggagttcaggcaccgcatcatgaaccctctgaccgaggatgagtttcagcagctgatcgccccatggatcgacatgttctatctgaatcacgccgagcaccccttcatgcagacaaagggcgtgaaggccaacgacgtgacccccatggagaagctgctggcaggcgtgtccggagcaacaaattgcgccttcgtgaaccagccaggacagggagaggccctgtgcggaggctgtaccgccatcgccctgtttaatcaggcaaaccaggcacctggattcggaggaggctttaagtctggactgaggggaggaaccccagtgaccacattcgtgagaggcatcgatctgaggagcacagtgctgctgaatgtgctgaccctgccacggctgcagaagcagtttcccaatgagagccacacagagaaccagcccacctggatcaagcctatcaagtctaacgagagcatccctgccagctccatcggcttcgtgagaggcctgttttggcagccagcccacatcgagctgtgcgaccccatcggcatcggcaagtgttcttgctgtggccaggaaagcaatctgaggtacaccggcttcctgaaggagaagttcacctttacagtgaacggcctgtggccccaccctcactctccatgtctggtgacagtgaagaagggcgaggtggaggagaagttcctggcctttaccacatccgccccctcttggacccagatcagcagagtggtggtggacaagatcatccagaacgagaatggcaacagagtggccgccgtggtgaatcagttcaggaacatcgccccacagtctcccctggagctgatcatgggcggctacaggaacaatcaggccagcatcctggagcggagacacgatgtgctgatgtttaatcagggctggcagcagtatggcaatgtgatcaacgagatcgtgacagtgggcctgggctacaagaccgccctgagaaaggccctgtatacattcgccgagggctttaagaacaaggacttcaagggagcaggcgtgagcgtgcacgagaccgccgagaggcacttttaccgccagtccgagctgctgatccccgatgtgctggccaatgtgaacttctcccaggccgacgaagtgatcgccgatctgagggacaagctgcaccagctgtgcgagatgctgtttaaccagtctgtggccccatacgcccaccaccccaagctgatcagcacactggccctggcaagggccaccctgtataagcacctgagggagctgaagccacagggaggaccttctaatgga

SEQ ID NO: 22 - DNA, 480 bp, Artificial Sequence
  Cas 11 gene
atggccgatgagatcgacgcaatggcactgtacagggcatggcagcagctggacaacggatcttgcgcacagatcaggcgcgtgagcgagcctgatgagctgagggacatcccagccttctatcggctggtgcagccctttggctgggagaatcctagacaccagcaggccctgctgaggatggtgttttgtctgagcgccggcaagaacgtgatccggcaccaggacaagaagagcgagcagaccacaggaatctccctgggacgcgccctggccaattccggccggatcaacgagcggagaatcttccagctgatcagggccgatcgcacagccgacatggtgcagctgaggcgcctgctgacccacgcagagcctgtgctggattggccactgatggcccgcatgctgacatggtggggcaagcgggagagacagcagctgctggaggacttcgtgctgaccacaaataagaacgcc

SEQ ID NO: 23 - protein, 888 aa, Artificial Sequence
  Cas 3 protein
MEPFKYICHYWGKSSKSLTKGNDIHLLIYHCLDVAAVADCWWDQSVVLQNTFCRNEMLSKQRVKAWLLFFIALHDIGKFDIRFQYKSAESWLKLNPATPSLNGPSTQMCRKFNHGAAGLYWFNQDSLSEQSLGDFFSFFDAAPHPYESWFPWVEAVTGHHGFILHSQDQDKSRWEMPASLASYAAQDKQAREEWISVLEALFLTPAGLSINDIPPDCSSLLAGFCSLADWLGSWTTTNTFLFNEDAPSDINALRTYFQDRQQDASRVLELSGLVSNKRCYEGVHALLDNGYQPRQLQVLVDALPVAPGLTVIEAPTGSGKTETALAYAWKLIDQQIADSVIFALPTQATANAMLTRMEASASHLFSSPNLILAHGNSRFNHLFQSIKSRAITEQGQEEAWVQCCQWLSQSNKKVFLGQIGVCTIDQVLISVLPVKHRFIRGLGIGRSVLIVDEVHAYDTYMNGLLEAVLKAQADVGGSVILLSATLPMKQKQKLLDTYGLHTDPVENNSAYPLINWRGVNGAQRFDLLAHPEQLPPRFSIQPEPICLADMLPDLTMLERMIAAANAGAQVCLICNLVDVAQVCYQRLKELNNTQVDIDLFHARFTLNDRREKENRVISNFGKNGKRNVGRILVATQVVEQSLDVDFDWLITQHCPADLLFQRLGRLHRHHRKYRPAGFEIPVATILLPDGEGYGRHEHIYSNVRVMWRTQQHIEELNGASLFFPDAYRQWLDSIYDDAEMDEPEWVGNGMDKFESAECEKRFKARKVLQWAEEYSLQDNDETILAVTRDGEMSLPLLPYVQTSSGKQLLDGQVYEDLSHEQQYEALALNRVNVPFTWKRSFSEVVDEDGLLWLEGKQNLDGWVWQGNSIVITYTGDEGMTRVIPANPK

SEQ ID NO: 24 - protein, 224 aa, Artificial Sequence
  Cas 5 protein
MRSYLILRLAGPMQAWGQPTFEGTRPTGRFPTRSGLLGLLGACLGIQRDDTSSLQALSESVQFAVRCDELILDDRRVSVTGLRDYHTVLGAREDYRGLKSHETIQTWREYLCDASFTVALWLTPHATMVISELEKAVLKPRYTPYLGRRSCPLTHPLFLGTCQASDPQKALLNYEPVGGDIYSEESVTGHHLKFTARDEPMITLPRQFASREWYVIKGGMDVSQ

SEQ ID NO: 25 - protein, 199 aa, Artificial Sequence
  Cas 6 protein
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 26 - protein, 363 aa, Artificial Sequence
  Cas 7 protein
MSNFINIHVLISHSPSCLNRDDMNMQKDAIFGGKRRVRISSQSLKRAMRKSGYYAQNIGESSLRTIHLAQLRDVLRQKLGERFDQKIIDKTLALLSGKSVDEAEKISADAVTPWVVGEIAWFCEQVAKAEADNLDDKKLLKVLKEDIAAIRVNLQQGVDIALSGRMATSGMMTELGKVDGAMSIAHAITTHQVDSDIDWFTAVDDLQEQGSAHLGTQEFSSGVFYRYANINLAQLQENLGGASREQALEIATHVVHMLATEVPGAKQRTYAAFNPADMVMVNFSDMPLSMANAFEKAVKAKDGFLQPSIQAFNQYWDRVANGYGLNGAAAQFSLSDVDPITAQVKQMPTLEQLKSWVRNNGEA

SEQ ID NO: 27 - protein, 502 aa, Artificial Sequence
  Cas 8 protein
MNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 28 - protein, 160 aa, Artificial Sequence
  Cas 11 protein
MADEIDAMALYRAWQQLDNGSCAQIRRVSEPDELRDIPAFYRLVQPFGWENPRHQQALLRMVFCLSAGKNVIRHQDKKSEQTTGISLGRALANSGRINERRIFQLIRADRTADMVQLRRLLTHAEPVLDWPLMARMLTWWGKRERQQLLEDFVLTTNKNA

SEQ ID NO: 29 - DNA, 960 bp, chicken
gacaactcagagttcaccatgggctccatcggtgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggtgagcctacagttaaagattaaaacctttgccctgctcaatggagccacagcacttaattgtatgataatgtcccttggaaactgcatagctcagaggctgaaaatctgaaaccagagttatctaaaagtgtggccacctccaactcccagagtgttacccaaatgcactagctagaaatcttgaaactggattgcataacttctttttgtcataaccattatttcagctactattattttcaattacaggttgttcgctttgataaacttccaggattcggagacagtattgaagctcaggtacagaaataatttcacctccttctctatgtccctttcctctggaagcaaaatacagcagatgaagcaatctcttagctgttccaagccctctctgatgagcagctagtgctctgcatccagcagttgggagaacactgttcataagaacagagaaaaagaaggaagtaacaggggattcagaacaaacagaagataaaactcaggacaaaaataccgtgtgaatgaggaaacttgtggatatttgtacgcttaagcaagacagctagatgattctggataaatgggtctggttggaaaagaaggaaagcctggctgatctgctggagctagattattgcagcaggtaggcaggagttccctagagaaaagtatgagggaattacagaagaaaaacagcacaaaattgtaaatattggaaaaggaccacatcagtgtagttactagcagtaagacagacaggatgaaaaat

SEQ ID NO: 30 - DNA, 23 bp, Artificial Sequence
  AI598 P1 Primer
acctgtggtgtagacatccagca

SEQ ID NO: 31 - DNA, 24 bp, Artificial Sequence
  AI831 P2 Primer
aaccgtgcagagaataagcttcat

SEQ ID NO: 32 - DNA, 32 bp, Artificial Sequence
  AI374 OVAS P3
ttgcctaaaaactgctcgtaatttactgttgt

SEQ ID NO: 33 - DNA, 20 bp, Artificial Sequence
  AI505GAPDH
tcaaatgggcagatgcaggt

SEQ ID NO: 34 - DNA, 21 bp, Artificial Sequence
  AI700GAPDH
agcaccagcaatccttttccc

SEQ ID NO: 35 - DNA, 579 bp, Artificial Sequence
  AI567 OVTs-hGMCSF-BGHpA
atgaagcttattctctgcacggttctgtcactggggatagctgccgtgtgcttcgccgctccaccgaaagcacccgcaagatctcctagcccatcaactcagccttgggaacatgttaatgcgattcaggaagcccgccgactgctgaacttgagccgggataccgcagccgagatgaatgagacagtagaagtgatctccgagatgttcgatttgcaagaacctacatgcctgcagacccgccttgagctgtacaaacagggactcaggggatccctgaccaagctcaaagggcccctcacgatgatggcttctcactacaaacagcactgtccacccactcctgagacaagctgcgcaacacagatcattactttcgagagtttcaaagaaaacttgaaggactttctcctggtcatcccgtttgactgttgggagccagtccaggaatgaaaaatcagcctcgactgtgccttctagttgccagccatctgttgtttgcccctcccccgtgccttccttgaccctggaaggtgccactcccactgtcctttcctaataaaatgaggaaattgcatc

SEQ ID NO: 36 - DNA, 8165 bp, Artificial Sequence
  AI567 full Sal-BamHI
gtcgaccttaagtcctcagacttggcaaggagaatgtagatttctacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttccctcatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattgttttgcattgtatggagctatgttttgctgtatcctcagaaaaaaagtttgttataaagcattcacacccataaaaagatagatttaaatattccagctataggaaagaaagtgcgtctgctcttcactctagtctcagttggctccttcacatgcacgcttctttatttctcctattttgtcaagaaaataataggtcacgtcttgttctcacttatgtcctgcctagcatggctcagatgcacgttgtacatacaagaaggatcaaatgaaacagacttctggtctgttactacaaccatagtaataagcacactaactaataattgctaattatgttttccatctccaaggttcccacatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggcgtcaaaggtcaaacttctgaagggaacctgtgggtgggtcacaattcaggctatatattccccagggctcagccagtgtctgtacatacagctagaaagctgtattgcctttagcactcaagctcaaaaggtaagcaactctctggaattaccttctctctatattagctcttacttgcacctaaactttaaaaaattaacaattattgtgttatgtgttgtatctttaagggtgaagtacctgcgtgataccccctataaaaacttctcacctgtgtatgcattctgcactattttattatgtgtaaaagctttgtgtttgttttcaggaggcttattctttgtgcttaaaatatgtttttaatttcagaacatcttatcctgtcgttcactatctgatatgctttgcagtttgcctgattaacttctagccctacagagtgcacagagagcaaaatcatggtgttcagtgaattctggggagttattttaatgtgaaaattctctagaagtttaattcctgcaaagtgcagctgctgatcactacacaagataaaaatgtggggggtgcataaacgtatattcttacaataatagatacatgtgaacttgtatacagaaaagaaaatgagaaaaatgtgtgtgcgtatactcacacacgtggtcagtaaaaacttttgaggggtttaatacagaaaatccaatcctgaggccccagcactcagtacgcatataaagggctgggctctgaaggacttctgactttcacagattatataaatctcaggaaagcaactagattcatgctggctccaaaagctgtgctttatataagcacactggctatacaatagttgtacagttcagctctttataatagaaacagacagaacaagtataaatcttctattggtctatgtcatgaacaagaattcattcagtggctctgttttatagtaaacattgctattttatcatgtctgcatttctcttctgtctgaatgtcaccactaaaatttaactccacagaaagtttatactacagtacacatgcatatctttgagcaaagcaaaccatacctgaaagtgcaatagagcagaatatgaattacatgcgtgtctttctcctagactacatgaccccatataaattacattccttatctattctgccatcaccaaaacaaaggtaaaaatacttttgaagatctactcatagcaagtagtgtgcaacaaacagatatttctctacatttatttttagggaataaaaataagaaataaaatagtcagcaagcctctgctttctcatatatctgtccaaacctaaagtttactgaaatttgctctttgaatttccagttttgcaagcctatcagattgtgttttaatcagaggtactgaaaagtatcaatgaattctagctttcactgaacaaaaatatgtagaggcaactggcttctgggacagtttgctacccaaaagacaactgaatgcaaatacataaatagatttatgaatatggttttgaacatgcacatgagaggtggatatagcaacagacacattaccacagaattactttaaaactacttgttaacatttaattgcctaaaaactgctcgtaatttactgttgtagcctaccatagagtaccctgcatggtactatgtacagcattccatccttacattttcactgttctgctgtttgctctagacaactcagagttcaccatgaagcttattctctgcacggttctgtcactggggatagctgccgtgtgcttcgccgctccaccgaaagcacccgcaagatctcctagcccatcaactcagccttgggaacatgttaatgcgattcaggaagcccgccgactgctgaacttgagccgggataccgcagccgagatgaatgagacagtagaagtgatctccgagatgttcgatttgcaagaacctacatgcctgcagacccgccttgagctgtacaaacagggactcaggggatccctgaccaagctcaaagggcccctcacgatgatggcttctcactacaaacagcactgtccacccactcctgagacaagctgcgcaacacagatcattactttcgagagtttcaaagaaaacttgaaggactttctcctggtcatcccgtttgactgttgggagccagtccaggaatgaaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctccatcggtgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggtgagcctacagttaaagattaaaacctttgccctgctcaatggagccacagcacttaattgtatgataatgtcccttggaaactgcatagctcagaggctgaaaatctgaaaccagagttatctaaaagtgtggccacctccaactcccagagtgttacccaaatgcactagctagaaatcttgaaactggattgcataacttctttttgtcataaccattatttcagctactattattttcaattacaggttgttcgctttgataaacttccaggattcggagacagtattgaagctcaggtacagaaataatttcacctccttctctatgtccctttcctctggaagcaaaatacagcagatgaagcaatctcttagctgttccaagccctctctgatgagcagctagtgctctgcatccagcagttgggagaacactgttcataagaacagagaaaaagaaggaagtaacaggggattcagaacaaacagaagataaaactcaggacaaaaataccgtgtgaatgaggaaacttgtggatatttgtacgcttaagcaagacagctagatgattctggataaatgggtctggttggaaaagaaggaaagcctggctgatctgctggagctagattattgcagcaggtaggcaggagttccctagagaaaagtatgagggaattacagaagaaaaacagcacaaaattgtaaatattggaaaaggaccacatcagtgtagttactagcagtaagacagacaggatgaaaaatagttttgtaaacagaagtatctaactactttactctgttcatacactatgtaaaacctactaagtaataaaactagaataacaacatctttctttctctttgtattcagtgtggcacatctgtaaacgttcactcttcacttagagacatcctcaaccaaatcaccaaaccaaatgatgtttattcgttcagccttgccagtagactttatgctgaagagagatacccaatcctgccagtaagttgctctaaaatctgatctgagtgtatttccatgccaaagctctaccattctgtaatgcaaaaacagtcagagttccacatgtttcactaagaaaatttctttttctcttgtttttacaaatgaaagagaggacaaataacatttctctatcaccgacctgaaactctacagtcttcagagaatgaatggcttgctaaaagaatgtcaaatcttaccatacagctatttcatattacactactaaatacactataaggcatagcatgtagtaatacactgtaaaatagctttttacactactatattattaatatctgttaattccagtcttgcatttcacatttgcaaaacgttttgaaattcgtatctgaaagctgaatactcttgctttacaggaatacttgcagtgtgtgaaggaactgtatagaggaggcttggaacctatcaactttcaaacagctgcagatcaagccagagagctcatcaattcctgggtagaaagtcagacaaatggtaaggtagaacatgctttgtacatagtgagagttggttcaccctaatactgagaacctggatatagctcagccagcgtgctttgcgttcaagcttaccagagctgttgtatgcctgttaagcagggcatacagtcatgaggctcttgaaaaatcttaacagacaaagggcaatggaaaatcggagttaagggatggtagggataaaatgcatagaaagaggtaccacgattttgatttttgccctaatgcctctctgcgtggttcctcaatttttctacttcattcctcatctcctcagagcattcctttccctcatgcttgaaacacagatgaaagactgtgaattctaactgagatgaaaacatccacaaccacacaacctctggtgtggagtcacattctgtgaaggcaaaaactaggccacgtaatctatgtgtgcaagctacgtgtaagctatgtgtgtgacaggacaatgtgaggaacatactatgtgcacaaggactgcagaataaacaggagcaaagtttttgaagaaaacagagtaaaatcccgttttcctcttttgttacattctttacatatatctcaaatttcctctttggttagaagcaagtaatatttatgtttcttggtactgtttgggttgaagaccattctgggataagagaaattccagtggttcttcccctaatcataaaatgtacaggtttagtttttttgtaacacagaaatctcttcatcttttatcttttgttgtgattctttatagagagagaaacaagacttactgacaatagcagcaagaaaatcaatcttggaagaacaagattgcagttgcaaaaacaaaccaatgtccttgcccctacatcctcttccccataaattctacattctctatctaccttgtgcttgccaacatgatatacgtaaactctcttttcctattcattcttaaaggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgaggtatatgggcataccttagagatgtaatctagaatttatgaagagagtagacatgttgttatatgaacactgcattagcgtatctgctcatttgtctgcatctctttcagacactgtgttaaaagcagggaattttccttatgtctctttcatcacaatattcctgacattgcaaagctcctgagaaataacttcagattcccacttttcctagggaggtcttcctggatgagaacaatcaatcatcttaactgtaactagatatttctgcatctaagaataatctttgttaaaactatattctctctctctttttttttttttttggttctccagcaagaaagcaaacctgtgcagatgatgtggatcc

SEQ ID NO: 37 - DNA, 36 bp, Artificial Sequence
  AI959OVAC3-Tg1-S
accgacagcaccaggacacaaataaataaggtgagc

SEQ ID NO: 38 - DNA, 36 bp, Artificial Sequence
  AI960OVAC3-Tg1-AS
acacgctcaccttatttatttgtgtcctggtgctgt

SEQ ID NO: 39 - DNA, 36 bp, Artificial Sequence
  AI961OVAC3-Tg2-S
accggtgagcctacagttaaagattaaaacctttgc

SEQ ID NO: 40 - DNA, 36 bp, Artificial Sequence
  AI962OVAC3-Tg2-AS
acacgcaaaggttttaatctttaactgtaggctcac

SEQ ID NO: 41 - DNA, 36 bp, Artificial Sequence
  AI963OVAC3-Tg3-S
accgattaaaacctttgccctgctcaatggagccac

SEQ ID NO: 42 - DNA, 36 bp, Artificial Sequence
  AI964OVAC3-Tg3-AS
acacgtggctccattgagcagggcaaaggttttaat

SEQ ID NO: 43 - DNA, 36 bp, Artificial Sequence
  AI965OVAC3-Tg4-S
accgtgtggccacctccaactcccagagtgttaccc

SEQ ID NO: 44 - DNA, 36 bp, Artificial Sequence
  AI966OVAC3-Tg4-AS
acacgggtaacactctgggagttggaggtggccaca

SEQ ID NO: 45 - DNA, 36 bp, Artificial Sequence
  AI981OVAC3-Tg5-S
accgctcaggtacagaaataatttcacctccttctc

SEQ ID NO: 46 - DNA, 36 bp, Artificial Sequence
  AI982OVAC3-Tg5-AS
acacgagaaggaggtgaaattatttctgtacctgag

SEQ ID NO: 47 - DNA, 36 bp, Artificial Sequence
  AI983OVAC3-Tg6-S
accgcaaaatacagcagatgaagcaatctcttagct

SEQ ID NO: 48 - DNA, 36 bp, Artificial Sequence
  AI984OVAC3-Tg6-AS
acacagctaagagattgcttcatctgctgtattttg

SEQ ID NO: 49 - DNA, 36 bp, Artificial Sequence
  AI985OVAC3-Tg7-S
accgcaatctcttagctgttccaagccctctctgat

SEQ ID NO: 50 - DNA, 36 bp, Artificial Sequence
  AI986OVAC3-Tg7-AS
acacatcagagagggcttggaacagctaagagattg

SEQ ID NO: 51 - DNA, 36 bp, Artificial Sequence
  AI987OVAC3-Tg8-S
accgaaaaacagcacaaaattgtaaatattggaaaa

SEQ ID NO: 52 - DNA, 36 bp, Artificial Sequence
  AI988OVAC3-Tg8-AS
acacttttccaatatttacaattttgtgctgttttt

SEQ ID NO: 53 - protein, 7 aa, Artificial Sequence
  NLS
PKKKRKV

SEQ ID NO: 54 - protein, 9 aa, Artificial Sequence
  NLS
PAAKRVKLD

SEQ ID NO: 55 - protein, 7 aa, Artificial Sequence
  NLS
PQPKKKP

SEQ ID NO: 56 - protein, 16 aa, Artificial Sequence
  NLS
KRPAATKKAGQAKKKK

SEQ ID NO: 57 - protein, 19 aa, Artificial Sequence
  NLS
KRTADGSEFESPKKKRKVE

SEQ ID NO: 58 - DNA, 35 bp, Artificial Sequence
  AI951 OVAC3Don-0S
acctgggtgcaatttacagcaccaggacacaaata

SEQ ID NO: 59 - DNA, 35 bp, Artificial Sequence
  AI952 OVAC3Don-0AS
tcctggtgctgtaaattgcacccaggtataccatg

SEQ ID NO: 60 - DNA, 35 bp, Artificial Sequence
  AI953 OVAC3Don-1S
acacaaataaattttgtgagcctacagttaaagat

SEQ ID NO: 61 - DNA, 35 bp, Artificial Sequence
  AI954 OVAC3Don-1AS
ctgtaggctcacaaaatttatttgtgtcctggtgc

SEQ ID NO: 62 - DNA, 35 bp, Artificial Sequence
  AI955 OVAC3Don-2S
agcctacagttatttattaaaacctttgccctgct

SEQ ID NO: 63 - DNA, 35 bp, Artificial Sequence
  AI956 OVAC3Don-2AS
aaaggttttaataaataactgtaggctcaccttat

SEQ ID NO: 64 - DNA, 35 bp, Artificial Sequence
  AI957 OVAC3Don-3S
agagttatctaattttgtggccacctccaactccc

SEQ ID NO: 65 - DNA, 35 bp, Artificial Sequence
  AI958 OVAC3Don-3AS
gaggtggccacaaaattagataactctggtttcag

SEQ ID NO: 66 - DNA, 35 bp, Artificial Sequence
  AI989 OVAC3Don-5S
gagacagtattgtttctcaggtacagaaataattt

SEQ ID NO: 67 - DNA, 35 bp, Artificial Sequence
  AI990 OVAC3Don-5AS
tctgtacctgagaaacaatactgtctccgaatcct

SEQ ID NO: 68 - DNA, 35 bp, Artificial Sequence
  AI991 OVAC3Don-6S
cctttcctctggtttcaaaatacagcagatgaagc

SEQ ID NO: 69 - DNA, 35 bp, Artificial Sequence
  AI992 OVAC3Don-6AS
tgctgtattttgaaaccagaggaaagggacataga

SEQ ID NO: 70 - DNA, 35 bp, Artificial Sequence
  AI993 OVAC3Don-7S
atacagcagatgtttcaatctcttagctgttccaa

SEQ ID NO: 71 - DNA, 35 bp, Artificial Sequence
  AI994 OVAC3Don-7AS
gctaagagattgaaacatctgctgtattttgcttc

SEQ ID NO: 72 - DNA, 35 bp, Artificial Sequence
  AI995 OVAC3Don-8S
agggaattacagtttaaaaacagcacaaaattgta

SEQ ID NO: 73 - DNA, 35 bp, Artificial Sequence
  AI996 OVAC3Don-8AS
tgtgctgtttttaaactgtaattccctcatacttt

SEQ ID NO: 74 - DNA, 35 bp, Artificial Sequence
  Donor 1
tttacagcaccaggacacaaataaataaggtgagc

SEQ ID NO: 75 - DNA, 35 bp, Artificial Sequence
  Donor 2
tttgtgagcctacagttaaagattaaaacctttgc

SEQ ID NO: 76 - DNA, 35 bp, Artificial Sequence
  Donor 3
tttattaaaacctttgccctgctcaatggagccac

SEQ ID NO: 77 - DNA, 35 bp, Artificial Sequence
  Donor 4
ttttgtggccacctccaactcccagagtgttaccc

SEQ ID NO: 78 - DNA, 35 bp, Artificial Sequence
  Donor 5
tttctcaggtacagaaataatttcacctccttctc

SEQ ID NO: 79 - DNA, 35 bp, Artificial Sequence
  Donor 6
tttcaaaatacagcagatgaagcaatctcttagct

SEQ ID NO: 80 - DNA, 35 bp, Artificial Sequence
  Donor 7
tttcaatctcttagctgttccaagccctctctgat

SEQ ID NO: 81 - DNA, 35 bp, Artificial Sequence
  Donor 8
tttaaaaacagcacaaaattgtaaatattggaaaa

SEQ ID NO: 82 - DNA, 100 bp, chicken
tagaaagctgtattgcctttagcactcaagctcaaaagacaactcagagttcaccatgggctccatcggtgcagcaagcatggaattttgttttgatgta

(19) United States
(12) Patent Application Publication
(10) Pub. No.: US 2023/0257768 A1
(43) Pub. Date: Aug. 17, 2023

(54) POULTRY CELL IN WHICH A GENE ENCODING A PROTEIN OF INTEREST IS KNOCKED-IN AT EGG WHITE PROTEIN GENE, AND METHOD FOR PRODUCING SAID POULTRY CELL

(71) Applicant: NATIONAL INSTITUTE OF ADVANCED INDUSTRIAL SCIENCE AND TECHNOLOGY, Tokyo (JP)

(72) Inventors: Isao OISHI, Kobe-shi, Hyogo (JP); Kyoko YOSHII, Ikeda-shi, Osaka (JP)

(73) Assignee: NATIONAL INSTITUTE OF ADVANCED INDUSTRIAL SCIENCE AND TECHNOLOGY, Tokyo (JP)

(21) Appl. No.: 18/009,187

(22) PCT Filed: Jun. 11, 2021
(86) PCT No.: PCT/JP2021/022359

§ 371 Date: Dec. 8, 2022

(30) Foreign Application Priority Data
Jun. 12, 2020 (JP) 2020-102405

(51) Int. Cl. C12N 15/85; A01K 67/027

(52) U.S. Cl. CPC C12N 15/8509; A01K 67/0275; A01K 2207/05; C12N 2310/20

(57) ABSTRACT

According to the present invention, a poultry cell knocked-in at an egg white protein gene, a knock-in method, a method for producing a knocked-in poultry cell and an egg or a poultry containing the knocked-in poultry cell may be provided.

### RELATED APPLICATIONS

This application claims the benefit of priority of Application No. 2020-102405 filed with the Japan Patent Office on Jun. 12, 2020. The contents of the priority application are incorporated herein by reference.

### TECHNICAL FIELD

This invention relates to a poultry cell knocked-in at an egg white protein gene, a knock-in method, a method for producing a knocked-in poultry cell, and an egg or a poultry comprising the knocked-in poultry cell.

### BACKGROUND ART

Attempts have been made to modify the genome in a poultry using genome editing techniques. Lentiviral vectors, TALEN, and the CRISPR Cas9 system have been reported for genome editing applied to a poultry (Non-Patent Literatures 1 and 2 and Patent Literature 1, respectively).

CRISPR-Cas systems are broadly classified into “Class 1” and “Class 2” in which the effectors that work in the process of cleaving DNA are composed of multiple Cas and a single Cas, respectively. The CRISPR-Cas9 system is widely known as a Class 2 CRISPR-Cas system. Class 1 CRISPR-Cas systems include Cas3 and the cascade complex (meaning the complex of cascade and crRNA. The same applies hereafter.) and others are known. In the CRISPR-Cas3 system, Cas3 (a protein with nuclease and helicase activities), cascade and crRNA cooperate to cleave DNA. Genome editing is difficult in eukaryotic cells when mature crRNAs are used, and efficient genome editing may be achieved by using pre-crRNAs, which are not usually used as a component of the system.

In the CRISPR-Cas3 system, crRNAs have been reported to recognize target sequences of 32-37 bases (Non-Patent Literature 3). On the other hand, crRNAs in the CRISPR-Cas9 system recognize target sequences of 18-24 bases in length. Therefore, the CRISPR-Cas3 system may recognize target sequences more accurately than the CRISPR-Cas9 system.

In eukaryotes, Class 2 CRISPR systems such as the CRISPR Cas9 system have become widely used as genome editing tools, but Class 1 CRISPR systems being composed of multiple Cas such as the CRISPR Cas3 system have been less developed. Patent Literature 2 disclosed that a use of the CRISPR Cas3 system successfully edited endogenous DNA regions in eukaryotes, but it is unclear whether the CRISPR Cas3 system would work in a poultry. Also, unlike the CRISPR Cas9 system, the CRISPR Cas3 system involves multiple molecules. Therefore, efficient genome editing methods in a poultry were unknown.

### PRIOR ART DOCUMENTS

### Patent Literatures

[Patent Literature 1] WO 2017/111144

[patent Literature 2] WO 2018/225858

### Non-Patent Literature

[Non-Patent Literature 1] Lillico, S. G. et al. Oviduct-specific expression of two therapeutic proteins in transgenic hens. Proc Natl Acad Sci U S A 104, 1771-1776 (2007)

[Non-Patent Literature 2] Park, T. S., Lee, H. J., Kim, K. H., Kim, J. S. & Han, J. Y. Targeted gene knockout in chickens mediated by TALENs. Proc Natl Acad Sci U S A. 111, 12716-12721 (2014).

[Non-Patent Literature 3] Ming Li et al., Nucleic Acids Res. 2017 May 5; 45(8): 4642-4654

### SUMMARY OF INVENTION

### Problem to be Solved by the Invention

A problem to be solved by the present invention is to provide a poultry cell knocked-in at an egg white protein gene, a knock-in method, a method for producing a knocked-in poultry cell, and an egg or a poultry comprising the knocked-in poultry cell.

### Means for Solving the Problem

The present invention provides:

[1] A poultry cell in which a gene encoding a protein of interest is knocked-in at an egg white protein gene, and wherein the cell comprises a deletion, substitution, or insertion in a DNA comprising a CRISPR Cas3 PAM sequence and a target sequence at 3′ side of the PAM sequence in the egg white protein gene as compared with the corresponding DNA region in wild type, wherein the egg white protein gene is selected from the group consisting of ovalbumin, ovomucoid, ovomucin, ovotransferrin, ovoinhibitor and lysozyme.
[2] The poultry cell of [1], wherein the DNA comprising the CRISPR Cas3 PAM sequence and the target sequence at 3′ side of the PAM sequence is 33-43 bases in length.
[3] The poultry cell of [2], wherein the DNA comprising the CRISPR Cas3 PAM sequence and the target sequence at 3′ side of the PAM sequence is 35 bases in length.
[4] The poultry cell of any one of the [1]-[3], wherein the DNA comprising the CRISPR Cas3 PAM sequence and the target sequence at 3′ side of the PAM sequence is selected from the group consisting of polynucleotides shown in SEQ ID NOs: 1-8.
[5] The poultry cell of [4], wherein the DNA comprising the CRISPR Cas3 PAM sequence and the target sequence at 3′ side of the PAM sequence is a polynucleotide shown in SEQ ID NO: 2.
[6] The poultry cell of any one of the [1]-[5], wherein the PAM sequence in the DNA comprising the CRISPR Cas3 PAM sequence and the target sequence at 3′ side of the PAM sequence is replaced with ttt.
[7] The poultry cell of any one of the [1]-[5], wherein the poultry cell is a primordial germ cell.
[8] An egg or a poultry comprising the poultry cell of any one of the [1]-[6].
[9] A method for producing a protein of interest, comprising a step of recovering the protein of interest from the egg of [8].
[10] A method of knocking-in a gene encoding a protein of interest into an egg white protein gene, comprising the step of introducing CRISPR Cas3 system and a donor construct into a poultry cell,
wherein the CRISPR Cas3 system comprises;
(a) CRISPR Cas3 protein or a polynucleotide encoding the CRISPR Cas3 protein,
(b) a cascade complex or a polynucleotide encoding the cascade complex, and
(c) crRNA for targeting the egg white protein gene or a nucleotide for expressing the crRNA,
wherein the egg white protein gene is selected from the group consisting of ovalbumin, ovomucoid, ovomucin, ovotransferrin, ovoinhibitor and lysozyme.
[11] The method of [10], wherein the crRNA for targeting the egg white protein gene comprises a polynucleotide selected from the group consisting of polynucleotides shown in SEQ ID NOs: 9-16.
[12] The method of [10] or [11], wherein the donor construct does not have at least one polynucleotide selected from the group consisting of polynucleotides shown in SEQ ID NOs: 1-8.
[13] A method for producing a knocked-in poultry cell in which a gene encoding a protein of interest is knocked-in at an egg white protein gene, comprising the step of introducing CRISPR Cas3 system and a donor construct into a poultry cell,
wherein the CRISPR Cas3 system comprises;
(a) CRISPR Cas3 protein or a polynucleotide encoding the CRISPR Cas3 protein,
(b) a cascade complex or a polynucleotide encoding the cascade complex, and
(c) crRNA for targeting the egg white protein gene or a nucleotide for expressing the crRNA,
wherein the egg white protein gene is selected from the group consisting of ovalbumin, ovomucoid, ovomucin, ovotransferrin, ovoinhibitor and lysozyme.
[14] The method of [13], wherein the crRNA for targeting the egg white protein gene is selected from the group consisting of polynucleotides shown in SEQ ID NOs: 9-16, or the nucleotide for expressing the crRNA is selected from the group consisting of polynucleotides shown in SEQ ID NOs: 1-8.
[15] The method of [13] or [14], wherein the donor construct does not have at least one polynucleotide selected from the group consisting of polynucleotides shown in SEQ ID NOs: 1-8.
[16] A kit for the method of any one of [10]-[15].

### Effect of the Invention

The present invention may provide a poultry cell knocked-in at an egg white protein gene, a knock-in method, a method for producing a knocked-in poultry cell, and an egg or a poultry comprising the knocked-in poultry cell.

### BRIEF DESCRIPTION OF DRAWINGS

FIG. 1 shows the design of the CRISPR Cas3 target sequence. Cas3 Tg1 is shown by SEQ ID NO: 1; Cas3 Tg2 is shown by SEQ ID NO: 2; Cas3 Tg3 is shown by SEQ ID NO: 3; Cas3 Tg4 is shown by SEQ ID NO: 4; Cas3 Tg5 is shown by SEQ ID NO: 5; Cas3 Tg6 is shown by SEQ ID NO: 6; Cas3 Tg7 is shown by SEQ ID NO: 7; Cas3 Tg8 is shown by SEQ ID NO: 8. Translation start site: capital ATG, double line: PAM sequence (aag), triangle: insertion site of a foreign gene in a donor vector, 5′HR: a homologous region at 5′ side of a donor vector, 3′HR: a homologous region at 3′ side of a donor vector, shaded area: polymorphism (T/C, A/G in the order 5′-3′).

FIG. 2 shows the design of a CRISPR Cas3 donor vector. The upper part of FIG. 2 shows a schematic of the exons and introns of the ovalbumin gene and a insertion site of a foreign gene. SEQ ID NO: 82 is part of exon 2. The middle part of FIG. 2 shows a schematic diagram of a donor vector used to construct a human GM-CSF heterologously knocked-in chicken by the CRISPR/Cas9 method. The lower part of FIG. 2 shows a schematic diagram of a donor vector used to construct a human GM-CSF knocked-in chicken by a CRISPR/Cas3 method.

FIG. 3 shows the results of a gene knock-in into a primordial germline genomic DNA after neomycin selection. The upper schematic shows, from top to bottom, the ovalbumin gene (top), a donor vector for a CRISPR/Cas3 method (middle), and a knocked-in state (bottom). Triangles P1-P3 schematically show primers. The bottom electrophoresis result shows a photograph in which the genome of CRISPR/Cas3/donor vector-introduced cells after the neomycin selection was amplified by PCR using P1/P2 and subjected to electrophoresis. The bottom graph shows the results of quantitative PCR.

FIG. 4 shows the results of gene knock-in into primordial germline DNA after neomycin repetitive selection. The upper schematic shows, from top to bottom, the ovalbumin gene (top), the donor vector for the CRISPR/Cas3 method (middle), and the knocked-in state (bottom). Triangles P1-P3 schematically show primers. The bottom electrophoresis result shows a photograph in which the genome of CRISPR/Cas3/donor vector-introduced cells after the neomycin selection was amplified by PCR using P1/P2 and subjected to electrophoresis. The bottom graph shows the results of quantitative PCR.

### DESCRIPTION OF EMBODIMENTS

In one aspect, the present invention relates to a poultry cell in which a gene encoding a protein of interest is knocked-in at an egg white protein gene, and wherein the cell comprises a deletion, substitution, or insertion in a DNA comprising a CRISPR Cas3 PAM sequence and a target sequence at 3′ side of the PAM sequence in the egg white protein gene as compared with the corresponding DNA region in wild type,

wherein the egg white protein gene is selected from the group consisting of ovalbumin, ovomucoid, ovomucin, ovotransferrin, ovoinhibitor and lysozyme.

In another aspect, the present invention relates to a method of knocking-in a gene encoding a protein of interest into an egg white protein gene, comprising the step of introducing CRISPR Cas3 system and a donor construct into a poultry cell,

wherein the CRISPR Cas3 system comprises;
(a) CRISPR Cas3 protein or a polynucleotide encoding the CRISPR Cas3 protein,
(b) a cascade complex or a polynucleotide encoding the cascade complex, and
(c) crRNA for targeting the egg white protein gene or a nucleotide for expressing the crRNA,
wherein the egg white protein gene is selected from the group consisting of ovalbumin, ovomucoid, ovomucin, ovotransferrin, ovoinhibitor and lysozyme.

In yet another aspect, the present invention relates to a method for producing a knocked-in poultry cell in which a gene encoding a protein of interest is knocked-in at an egg white protein gene, comprising the step of introducing CRISPR Cas3 system and a donor construct into a poultry cell,

wherein the CRISPR Cas3 system comprises;
(a) CRISPR Cas3 protein or a polynucleotide encoding the CRISPR Cas3 protein,
(b) a cascade complex or a polynucleotide encoding the cascade complex, and
(c) crRNA for targeting the egg white protein gene or a nucleotide for expressing the crRNA,
wherein the egg white protein gene is selected from the group consisting of ovalbumin, ovomucoid, ovomucin, ovotransferrin, ovoinhibitor and lysozyme.

The term “polynucleotide” herein refers to a polymerized nucleotides and is used synonymously with the terms “gene”, “nucleic acid” or “nucleic acid molecule”. Polynucleotides may be in the form of DNA (e.g., cDNA or genomic DNA) or RNA (e.g., mRNA). The term “protein” may be also used synonymously with “peptide” or “polypeptide”.

In the present specification, “knock-in” may occur homozygous or heterozygous in a chromosome. Therefore, an expression of a knocked-in target gene or the amount of a protein derived from said target gene is reduced or lost compared to the wild type. In a case where knocked-in is performed, an expression of a gene of interest or the amount of a protein derived from said gene of interest is increased compared to the wild type.

In the present specification, the “knocked-in” egg includes eggs produced from a female poultry having both heterozygous (+/−) genotype of, and fertilized eggs produced from a poultry having homozygous (+/+) genotype of a knock-in gene of interest. An egg produced from the female poultry having the homozygous (+/+) genotype of the knock-in gene of interest contains more expression or more expression products of a gene of interest than that of an egg produced from the female poultry having the heterozygous (+/−) genotype of the knock-in gene of interest.

The method of the present invention may also be applied to a cell with a modification. The cell, egg, or poultry obtained by the method may also be further modified. In one embodiment, the modification is genome editing.

The genome editing is a technique for a gene modification using a cleavage of double-stranded DNA and an error in repairing the cleavage and includes a nuclease capable of cleaving the target double-stranded DNA and a DNA recognition component that binds to or forms a complex with the nuclease. Examples of the genome editing technique include ZFN (zinc finger nuclease), TALEN, and CRISPR. For example, ZFN uses FokI (a nuclease) and a zinc finger motif (a DNA recognition component), TALEN uses FokI (a nuclease) and a TAL effector (a DNA recognition component), and CRISPR uses Cas9 (a nuclease) and a guide RNA (gRNA, a DNA recognition component) are widely used. The nuclease used in the genome editing is only required to have nuclease activity, and a DNA polymerase, a recombinase, and the like may be used other than the nuclease.

Examples of the poultry include a chicken, a quail, a turkey, a duck, a goose, a long-tailed cock, a Japanese bantam, a pigeon, an ostrich, a green pheasant, a guinea fowl, and the like. Preferable examples of the poultry include the chicken, the quail, and the like. The primordial germ cell may be from male or female. The primordial germ cell of the poultry such as a chicken is a floating cell and cultured in the presence of a feeder cell, such as a BRL cell and STO cell. Alternatively, the primordial germ cell may be cultured in the absence of the feeder cell by adding an appropriate cytokine in a medium.

A target gene to be modified by the genome editing is not limited, but is preferably an egg white protein gene. Egg white protein genes are those under the control of an egg white protein expression promoter, specifically ovalbumin, ovomucoid, ovomucin, ovotransferrin, ovoinhibitor, lysozyme, and the like.

In a case where a gene of interest is knocked-in by genome editing, the gene of interest is knocked-in at the locus of an egg white protein gene to preferably obtain an egg containing an expression product of the gene of interest instead of an expression product of the egg white protein gene. The “gene of interest” is a polynucleotide which encodes a protein to be expressed (called “protein of interest.”). The gene of interest may be a exogenous and endogenous gene, and a polynucleotide which encodes a desired protein may be selected as appropriate. The polynucleotide may be obtained using PCR, chemical synthesis, or other known techniques based on the sequence information. Examples of a protein as the expression product of the gene of interest include various secreted proteins and peptides, and specific examples thereof include a functional polypeptide, such as an antibody (a monoclonal antibody) or a fragment thereof (e.g., scFv, Fab, Fab′, F(ab′)2, Fv, a single-chain antibody, scFv, dsFv, etc.), an enzyme, a hormone, a growth factor, a cytokine (for example, GM-CSF or core region), an interferon, a collagen, an extracellular matrix molecule, and a vaccine, an agonistic protein, an antagonistic protein or a portion thereof, and the like. For example, in a case where the protein encoded by the gene of interest is a biologically active protein that may be administered to human as a medicine, such a protein is derived from a mammal, preferably from human. Further, in a case where the protein encoded by the gene of interest is an industrially applicable protein, such as a protein A and a protein constituting a spider thread, such a protein is derived from any organisms including a microorganism (bacteria, yeast, etc.), a plant, and an animal, or an artificial protein or the portion thereof.

The gene of interest may be a single gene or a plurality of genes. In a case where the gene is a plurality of genes, the plurality of genes are expressed under the control for the egg white protein gene. For example, the plurality of genes may be expressed by interposing a sequence such as IRES between the plurality of genes. Alternatively, the plurality of genes may be expressed by interposing a sequence encoding a 2A peptide or the like between the plurality of genes. In such a case, the plurality of genes is simultaneously expressed under the control of an ovalbumin promoter and the peptide is cleaved. In a case where the protein of interest is expressed, it may be designed to include an appropriate signal peptide (e.g., ovotransferrin signal peptide from chicken), or to include an additional sequence (e.g., polyA sequence) at the 3′ end, or codon usage may be changed to facilitate its expression in the poultry.

In a preferred embodiment of the knocked-in poultry egg of the present invention, the expression product of the gene of interest is dominantly expressed in the thick albumen. The term “dominant” herein refers to (a) a state in which an expression amount of the gene of interest in the thick albumen is 50% or more, 60% or more, 65% or more, 70% or more, 75% or more, 80% or more, 85% or more, 90% or more, 95% or more, or 98% or more by mass with respect to an expression amount of the foreign gene in a whole knocked-in egg or (b) a state in which an expression amount of the gene of interest in the thick albumen is 1.1 times or more, preferably 2 times or more, more preferably 10 times or more of an expression amount of the gene of interest in an egg other than the thick albumen in a relative concentration. The expression product of the knock-in gene of interest is concentrated in the thick albumen and may thus be easily purified. Further, the expression product of the gene of interest may be expressed in an active form. The thick albumen may become cloudy due to the expression product of the gene of interest. However, a cloudy protein may be easily solubilized by an ultrasonic treatment, adding a solubilizing agent such as arginine hydrochloride, or the like.

In a preferred embodiment of the present invention, the expression product of the knock-in gene expressed in the thick albumen may be in a soluble form or in an insoluble form. The expression product of the knock-in gene in the insoluble form may be purified as an active protein. The expression product of the knock-in gene is preferably purified after solubilized. For purification, a conventional purification method, such as a column and dialysis, may be used. As the genome editing, a zinc finger, TALEN, and CRISPR may be included. Of these, TALEN and CRISPR are preferable and CRISPR is more preferable. As a new genome editing method has been continuously developed, the present invention is not limited to the existing methods and any genome editing methods to be developed in the future may be used in the present invention.

When performing knock-in by the genome editing, it is preferable that a drug-resistant gene is stably integrated in the genome together with the useful gene of interest to select a knocked-in primordial germ cell by the drug-resistant gene. Examples of the drug-resistant gene include a neomycin-resistant gene (Neor), a hydromycin-resistant gene (Hygr), a puromycin-resistant gene (Puror), a blasticidin-resistant gene (blastr), a zeocin-resistant gene (Zeor), and the like. Of these, the neomycin-resistant gene (Neor) or the puromycin-resistant gene (Puror) is preferable.

When performing knock-in of the gene of interest at the egg white protein gene locus, a translation start site of the gene of interest is preferably coincided with a translation start site of the egg white protein gene. The gene of interest may be introduced into the primordial germ cell as single- or double-stranded nucleic acids. The double-stranded nucleic acids may be introduced in a form of a plasmid vector, a BAC (bacterial artificial chromosome) vector, or the like. A gene sequence around the translation start site of the egg white protein gene may be inserted immediately before the translation start site of the gene of interest to match the translation start site of the gene of interest with the translation start site of the egg white protein gene by the knock-in.

In a case where the gene of interest is knocked-in at the egg white gene locus to obtain a knocked-in chicken individual and its egg, it is desirable that the albumen of the egg is recovered to recover the product of the gene of interest. More desirably, a portion including the thick albumen surrounding the egg yolk is recovered to efficiently recover the product of the gene of interest.

In one embodiment of the present invention, genetically modified poultry may be produced by a conventional method from the genetically modified poultry primordial germ cell that is obtained by the gene modification method of the present invention. In a preferred embodiment, further, a (knocked-in) egg may be obtained from the genetically modified poultry. Specific procedures will be described below.

The genetically modified primordial germ cell is transplanted in a blastoderm, blood stream, or gonadal region of a recipient early embryo. Several hundreds to several thousands of the cells are transplanted by microinjection into the blood stream around the time of starting a blood circulation, preferably on the second or third day after the start of egg incubation. Further, the endogenous primordial germ cells of the recipient may be inactivated or reduced in number in advance by a drug or ionizing radiation before performing transplantation. The egg incubation is continued for the transplanted embryo according to a conventional method to obtain a transplanted individual. The transplantation and egg incubation may be performed in an ex-ovo culture system in which an eggshell is changed or a windowing method in which an eggshell is not changed. The hatched individual may be raised under a normal condition to sexual maturity to obtain a living individual (a chimeric individual). The chimeric individual is mated with a wild-type or genetically modified individual, or the genetically modified chimeric individual to produce a poultry offspring having the genetic modification derived from the transplanted cell. In a preferred embodiment, the primordial germ cell of the present invention obtained by the genome editing has high proliferation ability and differentiates into a large number of sperms or eggs having high fertilizability in the chimeric individual. In order to increase an efficiency of this process, a mating test may be performed after examining a frequency of gene modification in genomes of gametes or evaluating a contribution ratio of the transferred cells, or the offspring may be selected by a feather color. The genetically modified homozygous poultry may be obtained by mating the female chimeric poultry in which the genetically modified female primordial germ cells are transplanted with the male chimeric poultry in which the genetically modified male primordial germ cells are transplanted. Further, the present invention is not limited to internal fertilization of the poultry. In a case where a technique such as differentiating a primordial germ cell into a germ cell in vitro is developed in the future, the genetically modified poultry may be produced by artificial insemination or intracytoplasmic sperm injection using such a technique.

In another embodiment of the present invention, the genome editing may be performed without culturing the primordial germ cell. In such a case, the endogenous primordial germ cell is genetically manipulated by infecting the early embryo with various viral vectors or injecting a plasmid vector as a liposome complex into a blood stream of the early embryo to establish a chimeric individual and a recombinant offspring. The primordial germ cell obtained by the genome editing may include the gene modification at a high frequency and may have sufficiently high fertility to produce a recombinant poultry offspring or a genetically modified poultry offspring, and may be thus useful in the present embodiment. In one embodiment, the (endogenous) primordial germ cell may be genetically modified without culturing the primordial germ cell.

Examples of the viral vector used for gene manipulation by the genome editing include a retroviral vector, an adenoviral vector, an adeno-associated viral vector, a lentiviral vector, and the like. These viral vectors may be used for the genome editing both in the cultured primordial germ cell and the endogenous primordial germ cell. For example, in a case where the endogenous primordial germ cell is modified by the genome editing, the genome editing in the primordial germ cell may be performed by constructing a viral vector expressing a nuclease that recognizes and cleaves any target sequences and an sgRNA using a genome editing viral vector commercially available from various companies, processing the viral vector into an infectious form by packaging, and administering a resulting material into a place where the primordial germ cell exits, such as a blastoderm, blood stream, or gonadal region of the poultry early embryo. In this manner, a genetically modified individual and a genetically modified product may be obtained in the following generation. The commercially available genome editing viral vectors are offered by a number of companies worldwide, and examples thereof include an “AAVpro (registered trademark) CRISPR/Cas9 Helper Free System (AAV2)” available from Takara Bio Inc., which uses the adeno-associated viral vector, a “Lentiviral CRISPR/Cas9 System” available from System Biosciences, LLC, which uses the lentiviral vector, and the like. In a case where the gene modification involves knock-in, the viral vector required for the gene editing may be used with, for example, a viral vector, plasmid, Bac vector, or single- or double-stranded DNA including a gene to be knocked-in.

Further, a genome editing plasmid and a donor construct, either without or in combination with the viral vector, may be prepared in a cell membrane permeable form, such as a liposome complex, and administered into a place where the primordial germ cell exits, such as a blastoderm, blood stream, or gonadal region of the poultry early embryo, to perform the genome editing in the primordial germ cell and obtain a genetically modified individual and a genetically modified product in the following generation.

In one preferred embodiment, in the poultry egg from the knocked-in cell of the present invention obtained by the above method, an expression product of the gene of interest may be stably and highly expressed in the egg. Herein, “the expression product of the gene of interest being stably and highly expressed in the egg” means that a protein encoded by the gene of interest is expressed in an amount of about 1 mg or more per egg in each egg derived from different individuals. The expression amount of the protein of interest is preferably about 10 mg or more, more preferably 100 mg or more, per egg. Further, for example, in a case where the gene of interest is knocked-in at the chicken egg white protein locus, a product of the gene of interest (protein) is expressed in the thick albumen of the egg produced from the knocked-in female chicken in a concentration of 5 mg/ml. This concentration is much higher than that obtained by a conventional gene transfection method that does not rely on knock-in and thus causes random gene insertions. Since the gene of interest is inserted in an identical location, variation in expression level is small between individuals and in the same individual. Further, because the present invention uses a technique to perform knock-in at a translation start site of a gene that is actually expressing in a chicken individual, the gene expression is not reduced by an effect of gene silencing or the like in a G2 generation or later.

In the case where the gene of interest is knocked-in at the egg white protein locus by the present method, the product of the gene of interest (protein) in the egg produced from the knocked-in female chicken may be distributed to the thick albumen at a higher concentration than that distributed to the thin albumen. Thus, the product of the gene of interest may be efficiently recovered by recovering a portion including the thick albumen. Eggs deficient or reduced in egg white allergen protein may be obtained by raising chickens with a targeted knock-in of the egg white allergen gene using this method. Such an egg is expected to have low allergenicity.

The poultry egg from the knocked-in cell of the present invention is produced from a knocked-in poultry individual in which an identical gene of interest is inserted in an identical location in the whole body, thus the difference in a protein expression level between individuals is small and genetic information and character of the gene of interest may be properly transmitted over generations. Further, the main expression of the gene of interest may be restricted to an egg white by performing gene knock-in at a location of the egg white protein. In this manner, a possibility of affecting a development process and the health of chicken is clearly lower as compared to a case where the gene of interest is expressed in the whole body, which may make an excellent effect. In addition, it is further preferable that the gene of interest is expressed under the control for a gene that is highly expressing in the albumen, such as ovalbumin, to increase an expression efficiency of the gene of interest. In one embodiment of the invention, the knocked-in chicken may be efficiently established by the gene knock-in using genome editing. The gene of interest may be expressed in the egg white and the expression product of the gene of interest is accumulated in the albumen by using CRISPR Cas3 system. In another embodiment, the CRISPR Cas3 system may be used to make the product of the gene of interest more present in the thick albumen in the albumen. In yet another embodiment, the product of the gene of interest may be efficiently recovered by recovering a portion including the thick albumen from the egg containing the product of the gene of interest, in a preferred embodiment, from the egg in which the gene of interest is knocked-in at an albumen gene locus.

In one embodiment, the use of the CRISPR-Cas3 system may reduce off-target effects where unintended mutations are introduced, by precisely cleaving the target sequence. Reduced off-target effects may increase the efficiency of knocking-in a target sequence or may increase the efficiency of obtaining recombinants that do not have the unintended sequence. In a preferred embodiment, the CRISPR-Cas3 system may be used to efficiently obtain knocked-in chickens that produce the protein of interest in egg white, or to reduce the possibility of generation of knocked-in chickens with impaired development or health due to an unintended mutation.

In the CRISPR Cas3 system,

(i) a protein having nuclease activity and helicase activity,
(ii) a cascade complex, and
(iii) a crRNA,
cooperate to recognize the target sequence and cleave the DNA.

In the CRISPR Cas3 system, the protein having nuclease activity and helicase activity includes Cas3, and the cascade complex includes Cas5, 6, 7, 8 and 11. Each of these Cas protein groups (Cas3, 5, 6, 7, 8, and 11) may be introduced into cells as a protein or a polynucleotide encoding the protein independently or simultaneously with any selection of them. A skilled person in the art may appropriately prepare the concentration, amount, ratio, etc. of the Cas protein group so that it may function in the cell into which it has been transfected.

In the present invention, a nuclear localization signal may be added to the Cas protein group. The nuclear localization signal may be added to the N-terminal and/or C-terminal of the Cas protein group (5′ and/or 3′ end of the polynucleotide encoding each Cas protein group). The addition of nuclear localization signals may promote the localization of Cas protein group to the nucleus in the cell and improve genome editing efficiency. The nuclear localization signal may be any signal that may localize a protein into the nucleus, and any nuclear localization signal may be used as appropriate by a skilled person in the art. Specific examples of nuclear localization signals are, but not limited to, for example, PKKKRKV(SEQ ID NO: 53)( monopartite SV40), PAAKRVKLD(SEQ ID NO: 54)(c-myc), PQPKKKP(SEQ ID NO: 55)(p53), KRPAATKKAGQAKKKK(SEQ ID NO: 56)(nucleoplasmin), KRTADGSEFESPKKKRKVE(SEQ ID NO: 57)( binopartite SV40) preferably PKKKRKV(SEQ ID NO: 53) or KRTADGSEFESPKKKRKVE(SEQ ID NO: 57).

A PAM sequence in the CRISPR-Cas3 system are, for example, “AAG”, “AGG”, “GAG”, “TAC”, “ATG”, and “TAG”. In the present invention, the PAM sequence is preferably “AAG”. The target sequence is a sequence of 30, 31, 32, 33, 34, 35, 36, 37, 38, 39 or 40 bases in length adjacent to the 3′ side of the PAM sequence, prefereably 32-37 bases in length, more preferably 32 bases in length. Thus, the DNA copmrising the PAM sequence (3 bases) of CRISPR Cas3 and the target sequence at the 3′ side of the PAM sequence is 33-43 bases in length, preferably 35-40 bases in length. The DNA comprising the PAM sequence (3 bases) of CRISPR Cas3 and the target sequence at the 3′ side of the PAM sequence is preferably 35 bases in length.

In the present invention, it is contemplated that DNA comprising the target sequence at the 3′ side of the CRISPR Cas3 PAM sequence may also be targeted by the CRISPR Cas3 system if it contains one, two, three, or four deletion, substitution or insertion of bases compared to the wild-type sequence.

In a preferred aspect of a target sequence used in the present invention (including PAM sequences), the target sequence includes a polynucleotide selected from the group consisting of; Tg1:5′-aagacagcaccaggacacaAataaataaggtgagc-3′ (SEQ ID NO: 1), Tg2:5′-aaggtgagcctacagttaaagattaaaacctttgc-3′ (SEQ ID NO: 2), Tg3:5′-aagattaaaacctttgccctgctcaatggagccac-3′ (SEQ ID NO: 3), Tg4:5′-aagtgtggccacctccaactcccagagtgttaccc-3′ (SEQ ID NO: 4), Tg5:5′-aagctcaggtacagaaataatttcacctccttctc-3′ (SEQ ID NO: 5), Tg6:5′-aagcaaaatacagcagatgaagcaatctcttagct-3′ (SEQ ID NO: 6), Tg7:5′-aagcaatctcttagctgttccaagccctctctgat-3′ (SEQ ID NO: 7) and Tg8:5′-aagaaaaacagcacaaaattgtaaatattggaaaa-3′ (SEQ ID NO: 8). The “A” (capital A) in Tg1 indicates the polymorphism in chickens. The “A” may be “G”.

The CRISPR-Cas3 system may specifically recognize and cleave a target sequence by crRNA. A pre-crRNA is particularly preferred as crRNA in this invention. The pre-crRNA used in the present invention typically has the structure of “leader sequence-repeated sequence-spacer sequence-repeated sequence (LRSR structure)” or “repeated sequence-spacer sequence-repeated sequence (RSR structure).” The leader sequence is an AT-rich sequence and functions as a promoter to express a pre-crRNA. The repeated sequence is a sequence repeating with a spacer sequence in between, and the spacer sequence is a sequence designed in the present invention as a sequence complementary to the target DNA (originally it is a sequence derived from a foreign DNA incorporated in the course of adaptation). The pre-crRNA becomes a mature crRNA when cleaved by proteins constituting the Cascade (for example, Cas6 for types I-A, B, D-E and Cas5 for type I-C). Typically, the strand length of a leader sequence is 86 bases, and the strand length of a repeated sequence is 29 bases. The strand length of a spacer sequence is, for example, 10-60 bases, preferably 20-50 bases, more preferably 25-40 bases, typically 32-37 bases. Thus, in the case of the LRSR structure, the pre-crRNA used in the present invention has a strand length of, for example, 154-204 bases, preferably 164-194 bases, more preferably 169-184 bases and typically 176-181 bases. In addition, in the case of the RSR structure, the strand length is, for example, 68-118 bases, preferably 78-108 bases, more preferably 83-98 bases, typically 90-95 bases. In order to make the CRISPR-Cas3 system of the present invention function in eukaryotic cells, it is considered that the process is important by which the repeated sequences of a pre-crRNA are cleaved by the proteins constituting the Cascade. Thus, it should be understood that the above repeated sequences may be shorter or longer than the above strand length as long as such cleavage takes place. Specifically, it may be said that the pre-crRNA is a crRNA formed by adding sequences sufficient for cleavage by proteins constituting the Cascade to both ends of the mature crRNA described below. In this way, a preferred embodiment of the method of the present invention includes the step of cleaving a crRNA by proteins constituting the Cascade after introducing the CRISPR-Cas3 system into eukaryotic cells. On the other hand, the mature crRNA generated by cleavage of a pre-crRNA has a structure of “5′-handle sequence -spacer sequence-3′-handle sequence”. Typically, the 5′-handle sequence is composed of 8 bases from positions 22-29 of the repeated sequence and is held in Cas5. In addition, the 3′-handle sequence is typically composed of 21 bases from positions 1-21 in the repeated sequence, forms a stem loop structure with the bases of positions 6 to 21, and is held at Cas6. Thus, the strand length of a mature crRNA is usually 61-66 bases. Note that, since there are also mature crRNAs having no 3′-handle sequence depending on the type of the CRISPR-Cas3 system, the strand length is shortened by 21 bases in this case. Note that the sequence of an RNA may be appropriately designed according to the target sequence for which DNA editing is desired. In addition, the synthesis of RNA may be performed using any method known in the art.

One preferred aspect for crRNA used in the present invention comprises a polynucleotide selected from the group consisting of;

(SEQ ID NO: 9)
5′-aagacagcaccaggacacaAauaaauaaggugagc-3′,
(SEQ ID NO: 10)
5′-aaggugagccuacaguuaaagauuaaaaccuuugc-3′,
(SEQ ID NO: 11)
5′-aagauuaaaaccuuugcccugcucaauggagccac-3′,
(SEQ ID NO: 12)
5′-aaguguggccaccuccaacucccagaguguuaccc-3′,
(SEQ ID NO: 13)
5′-aagcucagguacagaaauaauuucaccuccuucuc-3′,
(SEQ ID NO: 14)
5′-aagcaaaauacagcagaugaagcaaucucuuagcu-3′
(SEQ ID NO: 15)
5′-aagcaaucucuuagcuguuccaagcccucucugau-3′
and
(SEQ ID NO: 16)
5′-aagaaaaacagcacaaaauuguaaauauuggaaaa-3′.
16).

In the present invention, a polynucleotide which expresses a crRNA-comprising polynucleotide may be used. The crRNA-expressing polynucleotide may be provided in a vector.

One preferred aspect for the Cas protein group used in the present invention is; Cas3; a protein encoded by a polynucleotide consisting of the base sequence shown in SEQ ID NO: 17 Cas5; a protein encoded by a polynucleotide consisting of the base sequence shown in SEQ ID NO: 18 Cas6; a protein encoded by a polynucleotide consisting of the base sequence shown in SEQ ID NO: 19 Cas7; a protein encoded by a polynucleotide consisting of the base sequence shown in SEQ ID NO: 20 Cas8; a protein encoded by a polynucleotide consisting of the base sequence shown in SEQ ID NO: 21; and Cas11; a protein encoded by a polynucleotide consisting of the base sequence shown in SEQ ID NO: 22.

One preferred aspect for the Cas protein group used in the present invention is; Cas3; a protein consisting of the amino acid sequence shown in SEQ ID NO: 23 Cas5; a protein consisting of the amino acid sequence shown in SEQ ID NO: 24, Cas6; a protein consisting of the amino acid sequence shown in SEQ ID NO: 25, Cas7; a protein consisting of the amino acid sequence shown in SEQ ID NO: 26, Cas8; a protein consisting of the amino acid sequence shown in SEQ ID NO: 27; and Cas11; a protein consisting of the amino acid sequence shown in SEQ ID NO: 28.

A polynucleotide encoding a wild-type protein which is composed in the CRISPR Cas3 system includes a polynucleotide which has been modified for efficient expression in eukaryotic cells. In other words, polynucleotides that encode a group of Cas proteins and have been modified may be used. One preferred aspect for polynucleotide modification is a modification to a base sequence suitable for expression in eukaryotic cells, for example, a codon may be optimized for an expression in eukaryotic cells.

A sequence having 70%, 80%, 90%, 95%, 99%, or more sequence identity with the sequences shown in the present invention may have the same function as the sequences shown in the SEQ ID NOs. Thus, for example, a Cas3 protein based on a sequence encoding Cas3 with 90% sequence identity to the sequence shown in SEQ ID NO: 17 still has nuclease and helicase activity.

When the CRISPR Cas3 system of the present invention was used to knock-in a gene encoding a protein of interest in an egg white protein gene, a DNA comprising a CRISPR Cas3 PAM sequence and a target sequence at 3′ side of the PAM sequence in the egg white protein gene may have a deletion, substitution, or insertion as compared with the corresponding DNA region in wild type. The deletion, substitution, or insertion may be derived from a donor construct in a process of an introduction into a poultry cell.

A donor construct may include a Homologous Recombination (HR) template in a HR repair mechanism and may optionally be incorporated into a vector. A homologous recombination repair is an intracellular mechanism for repairing single-stranded and double-stranded DNA damage. The HR template may comprise a polynucleotide encoding a gene of interest or an adjacent sequence that provides homology to DNA adjacent to the endogenous gene to be replaced. Such adjacent sequence includes upstream and/or downstream sequence of an endogenous gene. Preferably, the adjacent sequence includes upstream and downstream sequences of the endogenous gene. The adjacent sequence may be, but not limited to, any number of bases between about 250, 500, 750, 1000, 1500, 2000, 2500, 2800, 3000, 3500, 4000, 5000, 6000, 7000, 8000, 9000 and 10,000, respectively. Such adjacent sequence in the HR template may comprise any deletion, substitution or insertion provided that the homologous recombination repair mechanism is functional.

A donor construct optionally comprises a drug resistant gene. A drug resistant gene includes neomycin-resistant gene (Neor), hygromycin-resistant gene (Hygr), puromycin-resistant gene (Puror), blasticidin-resistant gene (blastr), zeocin-resistant gene (Zeor), etc. A drug resistant gene is preferrably Neomycin-resistant gene (Neor) or puromycin-resistant gene (Puror). As one specific aspect of the invention, the donor construct comprises the upstream DNA of the endogenous gene, a polynucleotide for the gene of interest and the downstream DNA of the endogenous gene in the order 5′ to 3′.

A donor construct optionally comprises a marker. A marker includes those that visualize the knocked-in cells, such as a fluorescent protein gene, and include but are not limited to EGFP, mCherry and dsRed.

In the CRIPR Cas3 system, the PAM sequence and the target sequence at the 3′ side of the PAM sequence are recognized, thereby shaving off a large amount of DNA around the target sequence (which may include the target sequence itself), so the donor construct used for knock-in preferably does not have the specific “PAM sequence and the target sequence at the 3′ side of the PAM sequence”. When the donor construct has a specific “PAM sequence and the target sequence at the 3′ side of the PAM sequence,” the donor construct itself may also be various unpredictable form due to a large deletion, or the knocked-in DNA may be recognized by the CRISPR Cas3 system again and be largely deleted. This makes it difficult to obtain a knocked-in cell in the expected state. As one specific aspect of the invention, the donor construct does not have at least one polynucleotide selected from the group consisting of polynucleotides shown in SEQ ID NOs: 1-8.

In the present invention, the phrase “do not have the PAM sequence and the target sequence at the 3′ side of the PAM sequence” is mutually convertible with “comprises a deletion, substitution, or insertion or a combination thereof in a DNA comprising a Cas3 PAM sequence and a target sequence at 3′ side of the PAM sequence as compared with the corresponding DNA region in wild type”. The phrase “do not have the PAM sequence and the target sequence at the 3′ side of the PAM sequence” means the absence of the PAM sequence and the target sequence at the 3′ side of the PAM sequence, as long as it is not recognized by the CRIPR Cas3 system, and includes any deletion, substitution or insertion or combinations thereof in the DNA comprising the PAM sequence of Cas3 and the target sequence at the 3′ side of the PAM sequence as compared with the corresponding DNA region in wild type. If a mutation occurs in the PAM sequence, for example, aag is replaced by ttt.

In the present invention, if it does not have the “PAM sequence and the target sequence at the 3′ side of the PAM sequence”, it differs by 1, 2, 3, 4, 5, 6, 7, 8, 9, 10, 15, 20, 25, 30, 35, or 40 bases or more as compared with the corresponding DNA region in wild type. The insertion may result in the fragmentation of the DNA comprising the Cas3 PAM sequence and the target sequence at 3′ side of the PAM sequence. The deletion or a substitution may result in the loss of the DNA comprising the Cas3 PAM sequence and the target sequence at 3′ side of the PAM sequence.

A cell comprising a sequence that does not have “the PAM sequence and the target sequence at 3′ side of the PAM sequence” may be obtained by preparing a DNA “comprising a deletion, substitution or insertion in a DNA comprising a Cas3 PAM sequence and a target sequence at 3′ side of the PAM sequence as compared with the corresponding DNA region in wild type” in the HR template and using it in a knock-in process.

A kit for the CRISPR-Cas3 system of the invention is a kit for knocking-in a gene encoding a protein of interest in an egg white protein gene, comprising a CRISPR Cas3 system and optionally a donor construct, wherein the CRISPR Cas3 system comprises;

(a) CRISPR Cas3 protein or a polynucleotide encoding the CRISPR Cas3 protein,
(b) a cascade complex or a polynucleotide encoding the cascade complex, and
(c) crRNA for targeting the egg white protein gene or a nucleotide for expressing the crRNA,
wherein the egg white protein gene is selected from the group consisting of ovalbumin, ovomucoid, ovomucin, ovotransferrin, ovoinhibitor and lysozyme.

The constituent elements of the kit of the present invention may be in an embodiment in which all or some of them are mixed, or may be in an embodiment in which each of them is independent. Other constituent elements of the kit of the invention may be selected by a skilled person in the art. The kits may include a variety of constitution for editing DNA in a poultry cell. The constituent elements of the kit may be capable of editing DNA of a poultry cell. The kit may also be provided with instructions for use.

In this specification, “about” means a range of ±10%, preferably ±5%.

The following examples are given below to illustrate the invention in specific and detailed terms, but the examples are used to illustrate the invention and are not intended to limit the invention.

### EXAMPLE 1

So far, a gene knock-in technology by genome editing has been used to knock-in a foreign gene in exon 2 of ovalbumin, the major protein of egg white, in an in-frame manner to realize mass production of foreign gene products in egg white (Patent Literature 1). However, CRISPR Cas3 is a new genome editing technology, and a new method had to be developed to mass-produce a foreign gene in egg white using CRISPR Cas3. In particular, since the target sequence and the DNA cleavage site are far apart in CRISPR Cas3 system, it was considered necessary to select an appropriate target site for efficient knock-in of the foreign gene in exon 2, especially near the translation start site.

### 1. Selection of Target Sites in Ovalbumin for CRISPR Cas3

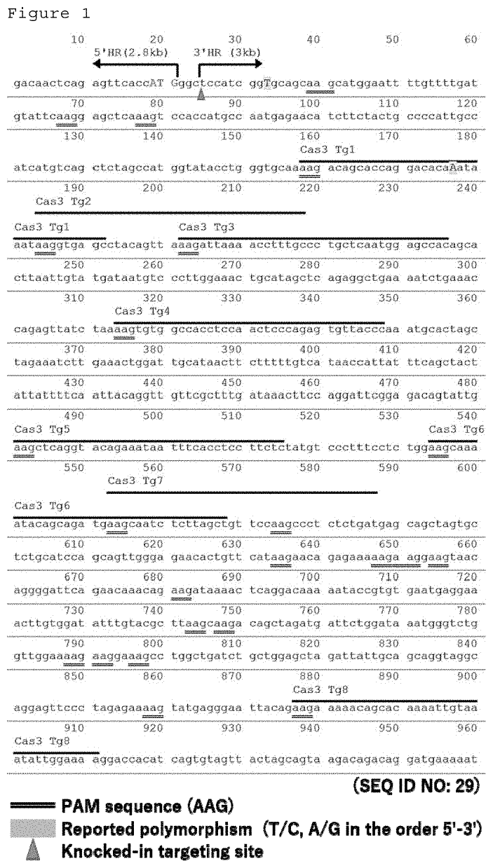

Eight target sequences, Tg1 to Tg8 shown in FIG. 1, between 138 and 893 bases downstream of the ovalbumin translation start site were selected based on the PAM sequence. The sequence shown in FIG. 1 (SEQ ID NO: 29) is derived from the chicken genome (highline species primordial germ cell). The polynucleotides with complementary sequences were obtained from Eurofin Genomics as polynucleotides with the following series base sequences to express crRNAs for targeting the respective target sequences; SEQ ID NO: 37 and SEQ ID NO: 38 corresponding to Tg1, SEQ ID NO: 39 and SEQ ID NO: 40 corresponding to Tg2, SEQ ID NO: 41 and SEQ ID NO: 42 corresponding to Tg3, SEQ ID NO: 43 and SEQ ID NO: 44 corresponding to Tg4, SEQ ID NO: 45 and SEQ ID NO: 46 corresponding to Tg5, SEQ ID NO: 47 and SEQ ID NO: 48 corresponding to Tg6, SEQ ID NO: 49 and SEQ ID NO: 50 corresponding to Tg7, SEQ ID NO: 51 and SEQ ID NO: 52 corresponding to Tg8. 100 pmol of the complementary polynucleotides for each target sequence were mixed, they were subjected to phosphorylation by T4 polynucleotide kinase (Toyobo) according to the accompanying instruction, and then annealed by heating 95° C. for 5 minutes and cooling to the room temperature. These were ligated into pBS-U6-crRNA-empty (addgene #134921 supplied from Mashimo, Osaka University) which was digested by BbsI (Thermo Fisher Scientific) using Ligation high ver.2 (Toyobo), and transformed into E. coli, and cloned a vector (pBS-U6OVATg1-Tg8) for expressing a crRNA which targets each target sequence from Tg1 to Tg8.

### 2. Construction of Knock-In Donor Vector

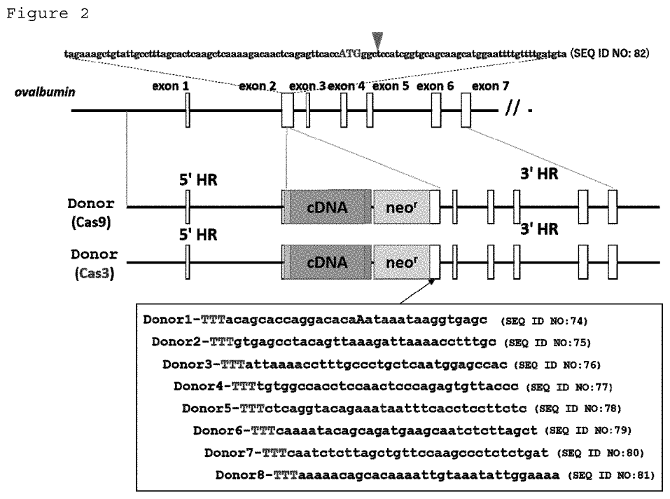

As shown in FIG. 2, donor vectors were constructed so that the donor vector comprises, in the order 5′ to 3′, (i) a 5′ homologous region 2.8 kb upstream from the ovalbumin translation start site, (ii) a foreign gene that is in-frame to the ovalbumin translation start site, (iii) a drug resistant gene, and (iv) a 3′ homologous region of 3 kb comprising 7 bases downstream from the ovalbumin translation start site. The foreign gene in (ii) was designed to link the human GM-CSF core region to the 3′ end of the chicken ovotransferrin signal peptide and to add a polyA sequence derived from the bovine growth hormone gene (SEQ ID NO: 35). The sequence of the insert portion of the donor vector linked from (i) to (iv) is shown in SEQ ID NO: 36. This was inserted into pBluescriptII (Clontech) between SalI-BamHI (pBS-OVA5-hGMCSF-neo-OVA3) and the following donor vector was constructed from this. Eight donor vectors (pBS-Donor 1-Donor 8) were constructed by replacing the PAM sequence (AAG) with TTT that corresponds to Tg1-Tg8 so that the 3′ homologous region is not recognized by the CRISPR Cas3 system. Specifically, we used pBS-OVA5-hGMCSF-neo-OVA3 as a template and obtained a primer set that is able to introduce the desired mutation (Eurofin Genomics) and performed site-specific mutagenesis by PCR. The polynucleotide sequences of the primers are Donor 1 (SEQ ID NO: 58 and SEQ ID NO: 59), Donor 2 (SEQ ID NO: 60 and SEQ ID NO: 61), Donor 3 (SEQ ID NO: 62 and SEQ ID NO: 63) , Donor 4 (SEQ ID NO: 64 and SEQ ID NO: 65), Donor 5 (SEQ ID NO: 66 and SEQ ID NO: 67), Donor 6 (SEQ ID NO: 68 and SEQ ID NO: 69), Donor 7 (SEQ ID NO: 70 and SEQ ID NO: 71) , and Donor 8 (SEQ ID NO: 72 and SEQ ID NO: 73). 20 ng of pBS-OVA5-hGMCSF-neo-OVA3 was used as a template, and 20 cycles of PCR (extension step was 72° C., 8 min) were performed using prime STAR HS (Takara) according to the protocol. After PCR reaction, 1 microliter of restriction enzyme DpnI (Toyobo) was added to digest the template plasmid overnight at 37° C., and then E. coli was transformed to construct the donor vectors (pBS-Donor 1-Donor 8) with a site-directed mutation.

### 3. Gene Knock-In by CRISPR Cas3 Using Chicken Primordial Germ Cells

Chicken primordial germ cells were cultured and transfected with crRNA expression vector (pBS-U6OVATg1-Tg8), a donor vector (pBS-Donor 1-Donor 8), CRISPR Cas3 expression vector (pPB-CAG-hCas3, Addgene#134920) (Supplied from Mashimo, Osaka University), and Cascade protein expression vector (pCAG-All-in-one-hCascade, Addgene #134919) (Supplied from Mashimo, Osaka University) using lipofectamine 2000 (Thermo Fisher Scientific). Specifically, according to Patent Literature 1, 1×10⁵ to 5×10⁵ chicken primordial germ line cells collected from day 2.5 embryo of White Leghorn (Hyline) and cultured, then 1.8 μg of pPB-CAG-hCas3, 0.6 μg of pCAG-All-in-one-hCascade, 0.6 μg of crRNA expression vector(pBS-U6OVATg1-Tg8), and 0.6 μg of the donor vector (pBS-Donor 1-Donor 8) corresponding to the crRNA expression vector were suspended in 300 μl of OPTI-MEM medium (Thermo Fisher Scientific) comprising 7 μl of lipofectamine 2000 and then added to the cultured primordial germ cells.

The medium was added with neomycin at a final concentration of 0.5 mg/ml from day 2 to day 7 after gene transfection to select for cells showing drug resistance. After neomycin was removed from the medium, the cells were cultured for about 1 month with appropriate replacement in feeder cells and medium, and genomic DNA was recovered from each. Each cell is hereinafter referred to as a Tg1/Donor 1-Tg8/Donor 8-transfected primordial germ cell.

PCR amplification was performed by Takara Mighty Amp 2.0 using 10 ng of genomic DNA as template and the following primers P1 and P2;

Primer P1: acctgtggtgtagacatccagca (SEQ ID NO: 30)
Primer P2: aaccgtgcagagaataagcttcat (SEQ ID NO: 31).

PCR amplification conditions were in accordance with the accompanying manual; after treatment at 95° C. for 2 minutes, 35 cycles of 95° C. for 10 seconds, 60° C. for 10 seconds, and 72° C. for 3 minutes were performed.

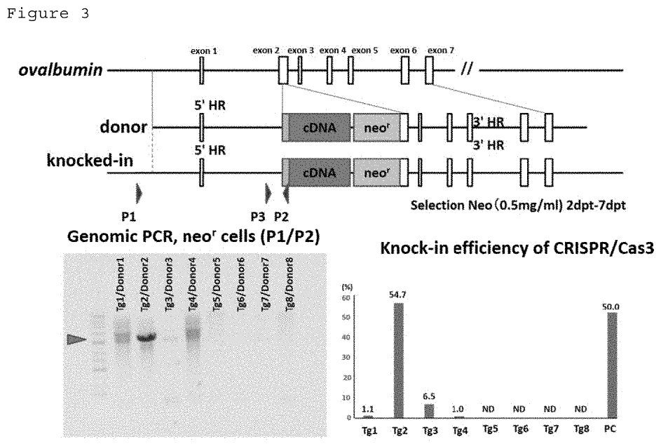

The amplified product was electrophoresed on 0.8% agarose is shown in the lower left of FIG. 3. The amplified products of the expected size of about 3.0 kb were obtained from genomic DNA derived from primordial germ cell transfected with Tg2/Donor 2, and the amplified products were also obtained from genomic DNA derived from primordial germ cell transfected with Tg1/Donor 1, Tg3/Donor 3, Tg4/Donor 4. On the other hand, almost no amplification products were observed in Tg5/Donor 5 -Tg8/Donor 8. This indicates that the Tg2/Donor 2 combination may efficiently knock-in a foreign gene at the ovalbumin translation start site in the chicken primordial germ cell when the CRISPR Cas3 system is used. It was also found that a combination of Tg1/Donor 1, Tg3/Donor 3, or Tg4/Donor 4 may be used to knock-in a foreign gene at the ovalbumin translation start site in the chicken primordial germ cell when the CRISPR Cas3 system is used.

### 4. Test of Efficiency by Quantitative PCR

Next, quantitative PCR was performed to examine the efficiency of donor vector knock-in into a primordial germ cell by the CRISPR Cas3 system. Primers P2 and P3 were used to identify the donor vector-derived regions. Primers P4 and P5 for the chicken glyceraldehyde-3-phosphate dehydrogenase (GAPDH) genome were used as internal controls. Primers P3-5 are

Primer P3:
(SEQ ID NO: 32)
TTGCCTAAAAACTGCTCGTAATTTACTGTTGT
Primer P4:
(SEQ ID NO: 33)
TCAAATGGGCAGATGCAGGT
Primer P5:
(SEQ ID NO: 34)
agcaccagcaatccttttccc.

The amplicon amplification efficiencies of the donor vector-derived region and GAPDH were tested by StepOnePlus real-time PCR system (Thermo Fisher Scientific) using THUNDERBIRD SYBR qPCR Mix (Toyobo). With respect to reaction conditions, reactions were performed in 20 μl reactions in accordance with the Toyobo manual and PCR was performed 40 cycles; two step cycle of 95° C. for 15 seconds and 60° C. for 1 minutes after treatment at 95° C. for 2 minutes.

With respect to a sample for genomic DNA, in addition to genomic DNA derived from primordial germ cells transfected with Tg1/Donor 1-Tg8/Donor 8, genomic DNA derived from human GM-CSF heterozygous knocked-in chicken (knocked-in at the ovalbumin translation start site) established by CRISPR/Cas9 method according to the method described in the Patent Literature 1 was used as control (PC). The Ct values of GAPDH and knock-in donor vectors obtained from each sample and PC (Ct(GAPDH) and Ct(GM-CSF), respectively) were used to calculate the relative amounts to the control heteroknocked-in chickens by the AACt method. Specifically, the knock-in efficiency of human GM-CSF in each cell transfected with Tg1/Donor 1-Tg8/Donor 8 was compared and quantified based on the following formula using PC which is a hetero knock-in as a calibrator and the GAPDH gene as an internal control.

Relative ratio=2\hat{ }(−ΔΔCt);

ΔCt(knocked-in cell)−ΔCt(PC)=ΔΔCt;

Ct(GMCSF(knocked-in cell))−Ct(GAPDH(knocked-in cell))=ΔCt(knocked-in cell);

Ct(GMCSF(PC))−Ct(GAPDH(PC))=ΔCt(PC).

As a result, a primordial germ cell genome from cell transfected with Tg1/Donor 1, Tg2/Donor 2, Tg3/Donor 3, and Tg4/Donor 4 showed 1.1%, 54.7%, 6.5%, and 1.0%, respectively, when the hetero knock-in genome was 50% (FIG. 3, lower right graph). The clear amplicons for donor vectors were not amplified in the primordial germ cell genome from cell transfected with the Tg5/Donor 5-Tg8/Donor 8. Consistent with the aforementioned results amplified with primers P1 and P2, the Tg2/Donor 2 combination was indicated to be suitable for knock-in using the CRISPR Cas3 system and capable of knocking-in a foreign gene about half of all ovalbumin alleles in a primordial germ cell under the conditions. It was also found that a combination of Tg1/Donor 1, Tg3/Donor 3, or Tg4/Donor 4 may be used to knock-in a foreign gene at the ovalbumin translation start site in a chicken primordial germ cell when the CRISPR Cas3 system is used.

### 5. Enhancement of Knock-In Efficiency in a Primordial Germ Cell After Selection by Repeated Drug Selection

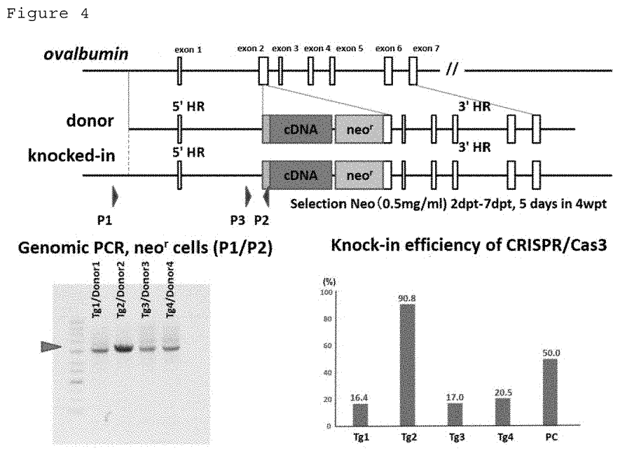

We examined whether the efficiency of knock-in in a primordial germ cell is enhanced by repeating a drug selection process. As in 3. above, Tg1/Donor 1-Tg4/Donor 4 were used to knock-in a foreign gene in a chicken primordial germ cell, and neomycin was added to the medium at a final concentration of 0.5 mg/ml from day 2 to day 7 after gene transection to select for cells showing drug resistance. After neomycin was removed from the medium, the cells were cultured for about 1 month, with appropriate replacement in feeder cells and medium. Neomycin was then added to the medium again at a final concentration of 0.5 mg/ml for 5 days for drug selection. After neomycin was removed from the medium again, the cells were cultured for about 3 weeks, with appropriate replacement in feeder cells and medium. Genomic DNAs were collected from each cells, PCR was performed using primers P1 and P2 as described in 3. above, and the amplified products were electrophoresed (FIG. 4, lower left). Compared to FIG. 3, the amplification products of Tg1/D1, Tg3/D3, and Tg4/D4 were clearer, indicating enhanced knock-in efficiency in primordial germ cells. As in 4. above, the quantitative PCR was also performed to calculate the donor relative amount with 50% of the genomic DNA from heterozygous knocked-in chickens. The results showed that a primordial germ cell genome from cell transfected with the Tg1/Donor 1, Tg2/Donor 2, Tg3/Donor 3, and Tg4/Donor 4 showed 16.4%, 90.8%, 17.0%, and 20.5%, respectively. It was considered that the knock-in efficiency was also enhanced in the cells as compared with the single drug selection from day 2 to day 7 after transfection as described in 3. above. Especially, it was considered that, in Tg2/Donor 2, the foreign gene was knocked-in in almost all alleles of ovalbumin. It is possible to establish knocked-in chickens that express large amounts of a foreign gene in an egg white via gonadal chimeric chickens by transplanting these cells into the early chicken embryo blood according to the method described in Patent Literature 1.

### INDUSTRIAL AVAILABILITY

The present invention provides a poultry cell knocked-in at an egg white protein gene, a knock-in method, a method for producing a knocked-in poultry cell and an egg or a poultry containing the knocked-in poultry cell.

1-16. (canceled)

17. A method of knocking-in a gene encoding a protein of interest into an egg white protein gene, comprising the step of introducing CRISPR Cas3 system and a donor construct into a poultry cell,
wherein the CRISPR Cas3 system comprises;
(a) CRISPR Cas3 protein or a polynucleotide encoding the CRISPR Cas3 protein,
(b) a cascade complex or a polynucleotide encoding the cascade complex, and
(c) crRNA for targeting the egg white protein gene or a nucleotide for expressing the crRNA, wherein the egg white protein gene is ovalbumin, and
(i) the crRNA for targeting the egg white protein gene comprises a polynucleotide shown in SEQ ID NO: 9, and the donor construct does not have the polynucleotide shown in SEQ ID NO: 1,
(ii) the crRNA for targeting the egg white protein gene comprises a polynucleotide shown in SEQ ID NO: 10, and the donor construct does not have the polynucleotide shown in SEQ ID NO: 2,
(iii) the crRNA for targeting the egg white protein gene comprises a polynucleotide shown in SEQ ID NO: 11, and the donor construct does not have the polynucleotide shown in SEQ ID NO: 3, or
(iv) the crRNA for targeting the egg white protein gene comprises a polynucleotide shown in SEQ ID NO: 12, and the donor construct does not have the polynucleotide shown in SEQ ID NO: 4.

18. The method of claim 17, wherein the crRNA for targeting the egg white protein gene comprises a polynucleotide shown in SEQ ID NO: 10, and the donor construct does not have the polynucleotide shown in SEQ ID NO: 2.

19. The method of claim 17, wherein the poultry cell is a primordial germ cell.

20. The method of claim 18, wherein the poultry cell is a primordial germ cell.

21. A method for producing a knocked-in poultry cell in which a gene encoding a protein of interest is knocked-in at an egg white protein gene, comprising the step of introducing CRISPR Cas3 system and a donor construct into a poultry cell,
wherein the CRISPR Cas3 system comprises;
(a) CRISPR Cas3 protein or a polynucleotide encoding the CRISPR Cas3 protein,
(b) a cascade complex or a polynucleotide encoding the cascade complex, and
(c) crRNA for targeting the egg white protein gene or a nucleotide for expressing the crRNA, wherein the egg white protein gene is ovalbumin, and
(i) the crRNA for targeting the egg white protein gene comprises a polynucleotide shown in SEQ ID NO: 9, and the donor construct does not have the polynucleotide shown in SEQ ID NO: 1,
(ii) the crRNA for targeting the egg white protein gene comprises a polynucleotide shown in SEQ ID NO: 10, and the donor construct does not have the polynucleotide shown in SEQ ID NO: 2,
(iii) the crRNA for targeting the egg white protein gene comprises a polynucleotide shown in SEQ ID NO: 11, and the donor construct does not have the polynucleotide shown in SEQ ID NO: 3, or
(iv) the crRNA for targeting the egg white protein gene comprises a polynucleotide shown in SEQ ID NO: 12, and the donor construct does not have the polynucleotide shown in SEQ ID NO: 4.

22. The method of claim 21, wherein the crRNA for targeting the egg white protein gene comprises a polynucleotide shown in SEQ ID NO: 10, and the donor construct does not have the polynucleotide shown in SEQ ID NO: 2.

23. The method of claim 21, wherein the poultry cell is a primordial germ cell.

24. The method of claim 22, wherein the poultry cell is a primordial germ cell.
